(19) United States
(12) Patent Application Publication
(10) Pub. No.: US 2025/0057412 A1
(43) Pub. Date: Feb. 20, 2025

(54) Smartphone Placido Disc Evaluation of Tear Film Break-Up

(71) Applicant: Mark Schneider, Corona, CA (US)

(72) Inventor: Mark Schneider, Corona, CA (US)

(21) Appl. No.: 18/234,299

(22) Filed: Aug. 15, 2023

(51) Int. Cl. A61B 3/10; A61B 3/107; A61B 3/14

(52) U.S. Cl. CPC A61B 3/101; A61B 3/107; A61B 3/14

(57) ABSTRACT

Improvements in capturing an image of a cornea with Placido's disk lines are disclosed. The imaging is with a clam shell clamping device that is easily clamped onto a cellular device and the lighting tube is centered on the camera so the image at the end of the tube can be captured. The clamping device includes a ring light source that illuminates the outside of the tube with an internally reflective cone. The tube has a plurality of geometrically spaced light and dark rings to create evenly spaced rings on the cornea. Imperfections in the cornea will distort the rings. The camera can capture the image and the image or picture can be forwarded to a doctor or other care giver to determine the perfection or imperfection of the cornea and tear film break-up device.

### STATEMENT REGARDING FEDERALLY SPONSORED RESEARCH OR DEVELOPMENT

Not Applicable

### CROSS REFERENCE TO RELATED APPLICATIONS

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

This invention relates to improvements in cornea imaging with a smartphone. More particularly, the present smartphone Placido disc evaluation of tear film break-up allows a person with a cellular phone to capture an image of their cornea to evaluate dry eye syndrome.

### Description of Related Art Including Information Disclosed Under 37 CFR 1.97 and 1.98

A number of patents and or publications have been made to address these issues. Exemplary examples of patents and or publication that try to address this/these problem(s) are identified and discussed below.

U.S. Pat. No. 4,993,826 issued on Feb. 19, 1991, to Paul R. Yoder Jr. and is titled Topography Measuring Apparatus. This patent discloses a contour measuring apparatus and method of using the same is disclosed to measure the three-dimensional contour of a surface. Structure is provided to direct first light beams onto the surface being measured. Reflections of the first light beams from the surface are received for generating electrical output signals corresponding to electro-optically measurable optical images.

U.S. Pat. No. 5,526,072 issued on Jun. 11, 1996, to Sami G. El Hage and is titled Apparatus and technique for automatic centering and focusing a corneal topographer. This patent discloses a corneal topographer including a directional light source, a CCD and a positioning stage all controlled by a computer for automatically centering and focusing the corneal image onto the CCD. The computer receives video signals from the CCD representing the corneal image, which is digitized and displayed on a real-time basis on a monitor screen. A reflection of the directional light source is positioned to align in the center of the corneal image. An operator moves an optics head to place the corneal image on the monitor screen, roughly focuses the image, and then commands the computer to take over.

U.S. Publication Number 2018/0092534 was published on Apr. 5, 2018, to Tareq Issam Nabhan and is titled System and Method for Ophthalmological Imaging Adapted to a Mobile Processing Device. This publication discloses a system and method for ophthalmological imaging is provided for use with a mobile processing device, wherein the mobile processing device comprises a camera lens, light source, and processor configured to process images captured, received, and/or delivered by the mobile processing device. The mobile processing device adapted ophthalmological instrument system comprises housing segments, circuitry, lights, and a frustum cone, wherein the frustum cone comprises reference lines of a plurality of circular, frusto-conical, alternating transparent and opaque concentric rings in the conical surface in optical alignment with the mobile processing device's camera lens and subject's central cornea and/or tear-film layer(s).

What is needed is a device that can be clamped onto most cell phones to capture images of the eye to determine tear film break-up. The proposed smartphone Placido disc evaluation of tear film break-up disclosed in this document provides the solution.

### BRIEF SUMMARY OF THE INVENTION

It is an object of the smartphone Placido disc evaluation of tear film break-up to easily be installed and removed onto a cell phone, mobile device or any device with an integrated camera.

It is an object of the smartphone Placido disc evaluation of tear film break-up to be able to transmit the images to a doctor or other person that can evaluate the stored images and to perform some initial evaluation from the images.

It is another object of the smartphone Placido disc evaluation of tear film break-up to include instructions for use and to provide real-time feedback from the information being collected.

It is another object of the smartphone Placido disc evaluation of tear film break-up is to include an integrated lighting system within a cone to provide consistency and even illumination.

It is another object of the smartphone Placido disc evaluation of tear film break-up to determine tear break-up film by processing the image from the camera.

It is another object of the smartphone Placido disc evaluation of tear film break-up to use the camera to determine roundness and concentricity of the curved image.

It is still another object of the smartphone Placido disc evaluation of tear film break-up to utilize a blocking tube that interrupts the light form the illumination source.

Various objects, features, aspects, and advantages of the present invention will become more apparent from the following detailed description of preferred embodiments of the invention, along with the accompanying drawings in which like numerals represent like components.

### BRIEF DESCRIPTION OF THE SEVERAL VIEWS OF THE DRAWING(S)

FIG. 1 shows a top perspective view of the smartphone Placido disc evaluation of tear film break-up.

FIG. 2 shows a bottom perspective view of the Placido imaging for a cell phone.

FIG. 3 shows a side view of the Placido imaging for a cell phone.

FIG. 4 shows a chart of pupil size based upon light intensity.

FIG. 5 shows a chart of ring height based upon distance from the lens.

FIG. 6 shows a tube with rings that reflect from the cornea into the camera of a cell phone to create a Placido image.

FIG. 7 shows the Placido image from a cell phone camera looking through the tube.

FIG. 8 shows the resulting image reflected of the cornea after rotation and color correction.

FIG. 9 shows an enlarged image from FIG. 8, centered with a grid overlaid onto the image.

FIG. 10 shows an enlarged image from FIG. 8 converted to a black and white image with processing.

FIG. 11 shows the image from FIG. 9 with fewer angles of resolution with quadrant references.

FIG. 12 shows the image from FIG. 11 masked and converted from Cartesian to a polar graph.

FIG. 13 shows graph from FIG. 12 converted to lines for processing.

FIG. 14 shows the graph from FIG. 13 with block processing.

FIG. 15 shows the block processing from FIG. 14 converted from Polar to Cartesian to show the imperfections in the cornea.

FIG. 16 shows the initial cornea image.

FIG. 17 shows the changed cornea image with tear film break-up.

FIG. 18 shows the tear film break-up after processing.

### DETAILED DESCRIPTION OF THE INVENTION

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention but is merely representative of various embodiments of the invention. The illustrated embodiments of the invention will be best understood by reference to the drawings, wherein like parts are designated by like numerals throughout.

Item Numbers and Description

| | |
|---|---|
| 12 camera | 14 eye |
| 15 lens offset | 16 cornea diameter |
| 17 cornea | 18 pupil size |
| 19 cell phone | 20 Placido imaging device |
| 21 upper clam shell | 22 lower clam shell |
| 23 hinge | 24 step |
| 25 adjustable opening | 26 opening |
| 27 pad | 30 light ring |
| 31 switch | 32 light ray(s) |
| 33 outer ring projection | 34 inner ring projection |
| 35 lens center of curvature | 40 tube |
| 41 eyepiece | 42 rings |
| 43 target | 44 camera opening |
| 45 eyepiece | 46 diameter |
| 51 image | 52 tear meniscus |
| 53 grid | 97 in and out |
| 98 push | 99 close |

Several issues need to be resolved to provide a consistent reliable image so an average person with a cell phone can take an acceptable image. The multitude of cell phones with different cameras and different camera locations presents a first issue to be resolved. Nearly all cell phones and cellular devices have two cameras, one higher resolution camera in the rear of the phone and one lower resolution phone in the front of the phone. The low-resolution camera in the front of the phone is usually placed near an outer edge of the screen, while the higher resolution camera can be located in any position, including the center back of the cell phone. While making a device to accommodate any camera location can be accomplished by increasing the size of the proposed design, using the front facing low resolution camera on the edge of the phone will provide an image of sufficient resolution to determine the roundness of the cornea.

The second issue is providing even lighting to eliminate or overcome ambient light. In the preferred embodiment the cornea is illuminated with the equivalent of a ring light that sends light in one direction to the cornea. Testing has identified that the cell phone camera determines focus location and brightness based upon locating an essentially flat image at a distance from the lens of the cell phone. Proper illumination of the cornea at a distance within the focal length of the cell phone camera results in cell phone camera finding the surface of the cornea, setting the focus length to the cornea and identifying the optimal contrast for a consistent image. This is all performed within most cell phones with the cell phone cornea Placido imaging disclosed in this document. The brightness of the illumination can have more than one level of illumination. The different levels of illumination can be adjusted based upon eye color and/or dilation of the pupil.

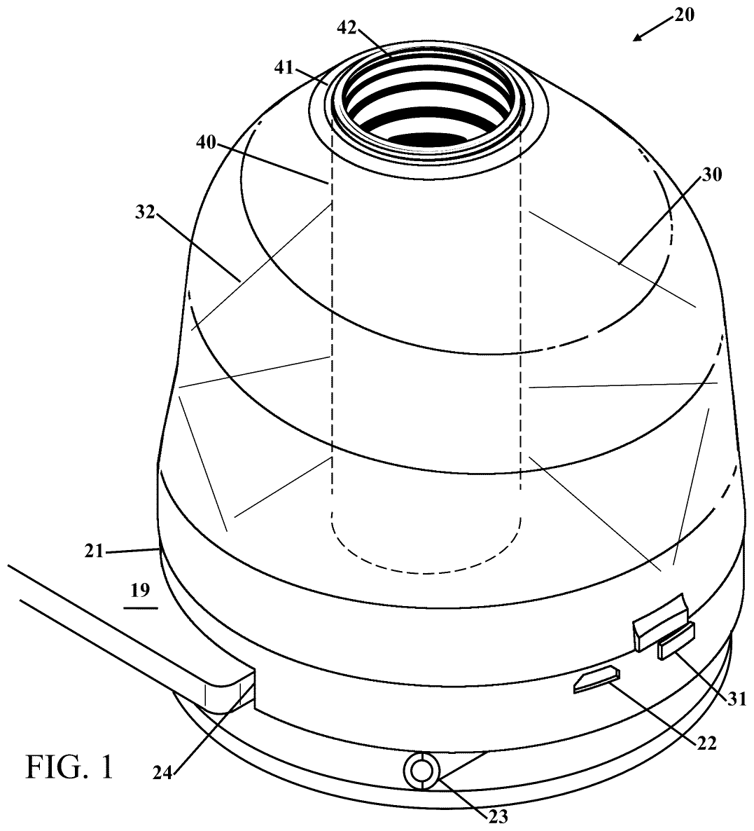

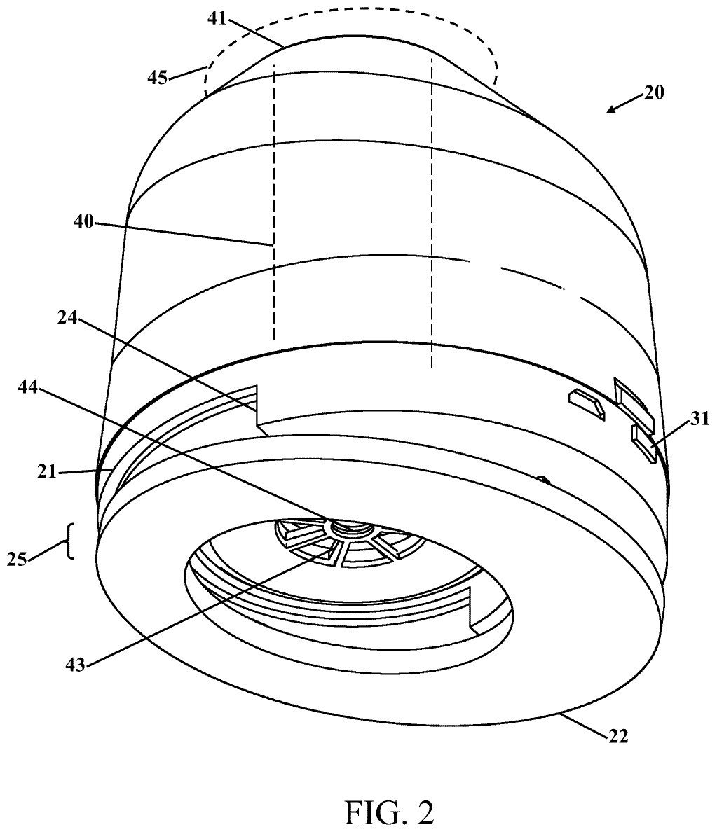

FIG. 1 shows a top perspective view of the Placido imaging device 20 on a cell phone 19 and FIG. 2 shows a bottom perspective view of the Placido imaging device 20 for a cell phone. The Placido imaging device 20 clamps onto a cell phone 19 in a clam shell design where there is an upper clam shell 21 portion that hinges with a hinge 23 mechanism on a lower clam shell 22. In this embodiment there is a step 24 that limits how far the Placido imaging device 20 can be placed over the cell phone 19 and provides a location for batteries that power illumination of the Placido imaging device 20.

The upper clam shell 21 and the lower clam shell 22 hinge to open the clam shell to accept the cell phone therein between. The clam shell portions allow the device to be easily installed, positioned, and removed from the cell phone. They further allow for easy adjustment and position on nearly all cell phones, tablets, and other similar mobile devices with a camera. Generally, most cell phones have a thickness of between 0.35 and 0.2 inches in thickness and the Placido imaging device 20 accommodates the different thicknesses in the clam shell adjustable opening 25.

In this embodiment the Placido imaging device 20 has an illumination ring type light source or light ring 30 that transmits light into a reflective cone so light is reflected evenly within the reflective cone and onto the tube 40 that is surrounded by the reflective cone. Testing has identified that the light ring 30 provides a more consistent and even illumination, but in some ambient lighting conditions the rings 42 can provide an image on the cornea. The light ring 30 has a switch 31 that can provide a single level of illumination or multiple levels of illumination. The light ring 30 has a sufficient number of lights to provide even illumination onto the tube 40. An enclosure around the tube is internally reflective to evenly distribute the light to the tube 40. The light 30 passes through the tube 40. Within the tube 40 are light and dark bands that cast an image onto the cornea when an eye is placed on the eyepiece 42.

In FIG. 2 the bottom surface of the lower clam shell 21 is visible. The bottom surface shows a target 43 and a camera opening 44. These features provide three functions. First, it assists a person to locate the camera lens in the center of the target. Second, the open nature of the target 43 allows a person to see a larger amount of the phone to make it easier to determine the position of the cell phone camera relative to the central camera opening. Third, it provides a focal point for the user to determine where to look through the end of tube 40. In an ideal situation the cell phone camera lens needs to be centered in the tube 40 and the eye of the user needs to look straight into the lens of the camera. In these conditions the only limit to a good picture is the movement of the user when the picture is taken. Some other variables can be the user placing the eye in a position that is not centered in the eyepiece 40. Typically, a non-centered eye is visible in the picture. While a round eyepiece is shown it is also contemplated that the eyepiece 45 can be contoured to assist the user to center the eye within the tube 40.

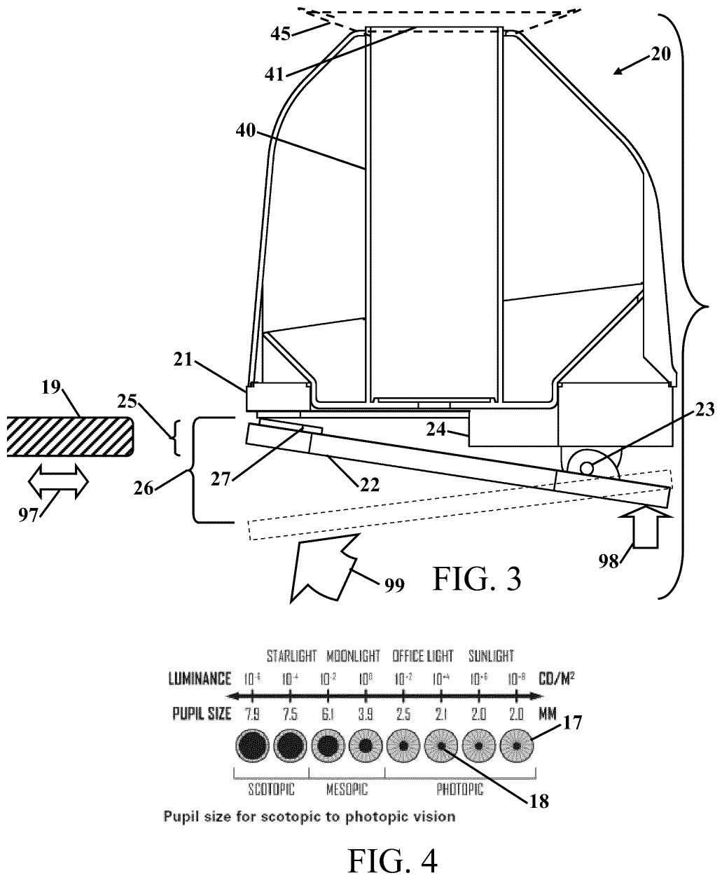

FIG. 3 shows a side view of the Placido imaging for a cell phone 19. To install a cell phone 19 the back of the two clam shells 21/22 and squeezed to pivot the hinge 24 to open the opposing side of the clam shell. A spring in the hinge 23 operates to close the clam shell and clamp an object, such as a cell phone 19 therein between 25. The opening 26 is the clam shell housing can accommodate a variety of thickness of cell phones 19 and can also accommodate cell phones 19 that are installed in a case without removing the case to use the Placido imaging device 20. Once the clam shells are opened the cell phone 19 can be moved in, out 97, up to the stop 24 or side-to-side to center the camera within the tube 40. One or a plurality of pads 27 frictionally hold the position of the Placido imaging device 20 on the cell phone 19.

FIG. 4 shows a chart of pupil size based upon light intensity. Optimal image of the cornea image 17 is obtained when the pupil size is properly obtained. In the preferred embodiment the optimal lighting to the eye is between office lighting and sunlight. The ring light intensity along with the amount of light that passes into the tube 40 is set based upon the light intensity, material properties of the tube 40 and the thickness of the materials that are used.

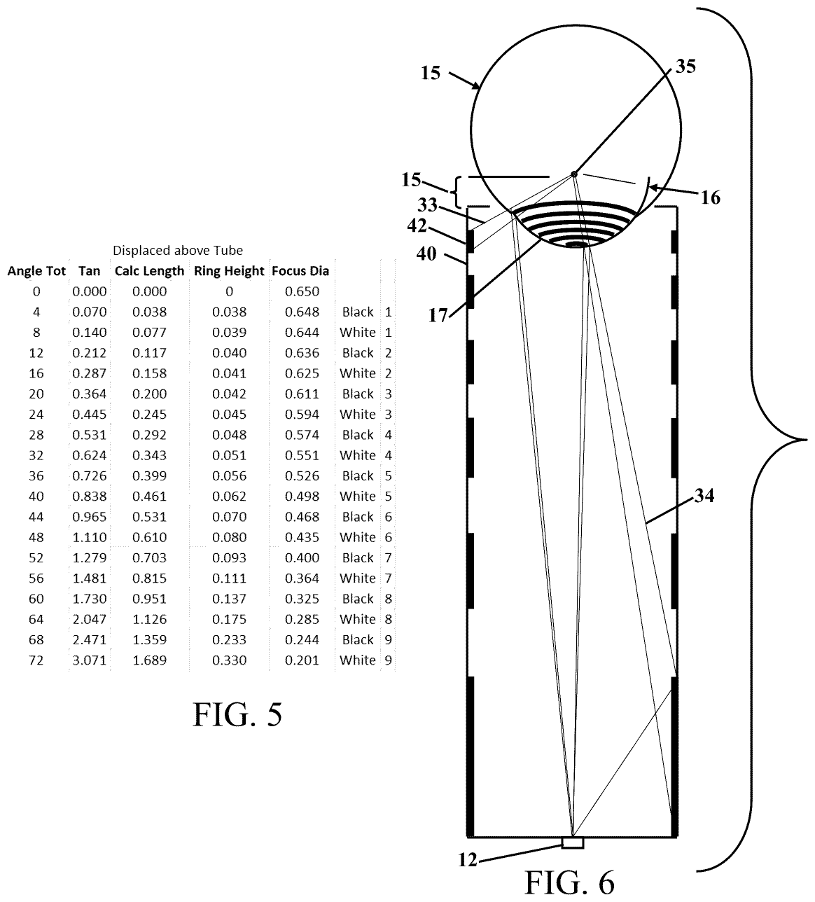

FIG. 5 shows a chart of ring height based upon distance from the lens and FIG. 6 shows a tube with rings that reflect from the cornea into the camera of a cell phone to create a Placido image. To project a consistent and even spacing of light and dark rings 42 onto the cornea 17. The geometry and relationship of camera 12, tube 40 and cornea 17 are determined. While every eye 15 and cornea 17 can be different, the eye is generally about 1 inch in diameter and the diameter 16 cornea 17 is about 0.7 inches. Using a tube diameter of 1 inch the lens center of curvature 35 of the cornea is located at a distance of about 0.15 inches above the upper rim of the tube 40. Using this relationship, the location, and thicknesses of the rings 42 can be calculated as shown in the table figure.

The height of the tube 40 is determined based upon the minimum focus distance for the camera 12. While a focus distance of 2 inches or less is possible with some cell phone cameras, a tube length of 3 inches is sufficient for the front camera of most cell phones and mobile devices with cameras. The outer ring projection 33 and the inner ring projection 34 is shown in the figure being projected through the tube 40, onto the cornea 17 as imaged in the camera 12.

In the table a step angle of 4 degrees is used to project (essentially) 9 light and dark rings but using a different step angle can form more or less rings using the same tube diameter and length. Adjusting the distance from the lens offset 15 will also cause changes to the height and location of the rings 42. While a conical, elliptical, square or other shape tube 40 can be used, the ring 42 geometry can be calculated. In the preferred embodiment the rings 42 are evenly spaced light and dark solid rings, the rings can be created to form an image at most of the visible cornea surface. In addition, the projected image can be dots, dashes, or other even words. Because the preferred embodiment is a round tube 40, the rings can be printed on film or other transparent or translucent material and can be interchangeably inserted into the inside diameter 46 of the tube 40 as show in FIG. 8.

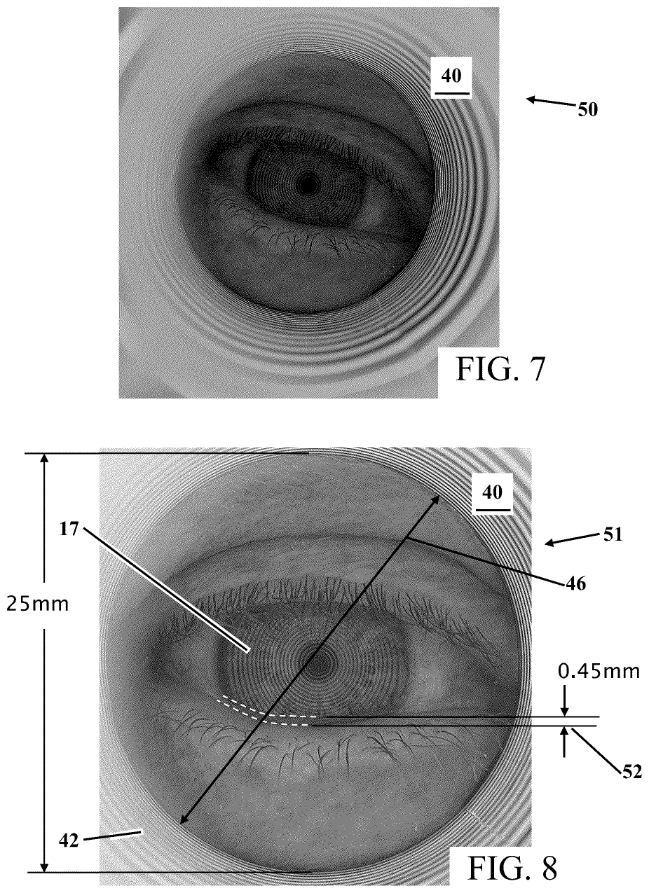

FIG. 7 shows the Placido image from a cell phone camera looking through tube 40. This picture is a typical image that a user can obtain using their smartphone. The eye can be rotated, and the picture resolution is not known. As smartphone camera technology changes the resolution will change. From this image the application will correct the color and rotate the image. FIG. 8 shows the resulting image 51 of the rings 42 reflected off the cornea 17 after rotation and color correction. In this image about 20 rings 42 are shown with a ring step of 2 degrees per ring change. Because the resolution of the camera is unknown, the diameter of the tube 46 (as shown in FIG. 8) is used for measurement references. The tube 40 diameter 46 in this picture is 25 mm. The application will prompt the user to blink several times to lubricate the cornea 17. The application detects the tear meniscus 52 and scales the measurement as 0.45 mm. The tear meniscus typically is 0.1 to 0.5 mm. When the cornea is wet the rings will reflect. An initial image is acquired, and the roundness of the eye can be measured.

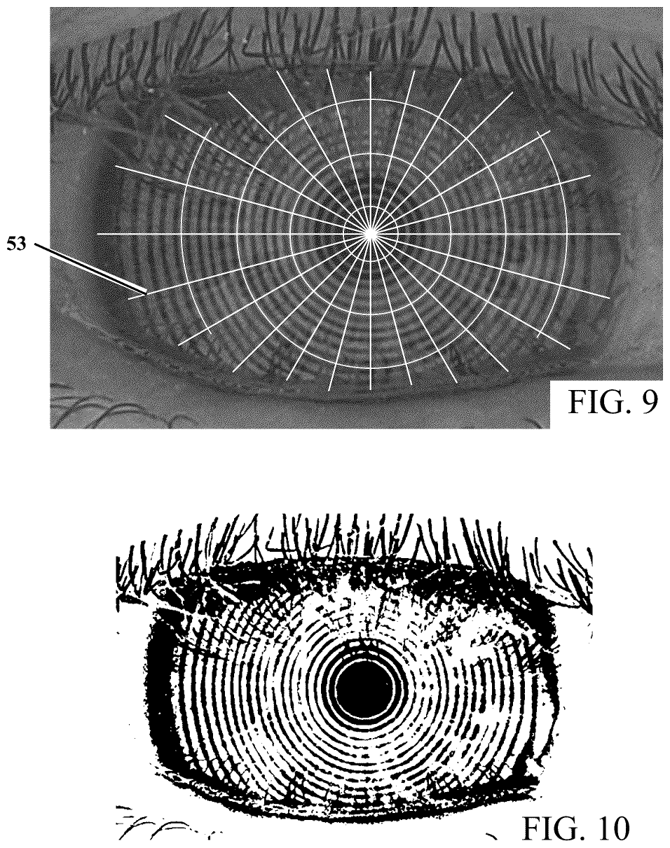

FIG. 9 shows an enlarged image from FIG. 8, centered with a grid 53 overlaid onto the image. The grid 53 establishes a reference orientation. The image can be further processed to define the edges of the reflected rings. FIG. 10 shows an enlarged image from FIG. 8 converted to a black and white image with processing. The captured image can then be processed with filters that turn the color image into black and white rings that define the edges of the rings. Further processing can also intemperate the roundness of the rings and the concentricity of the rings to determine if further action can or should be taken.

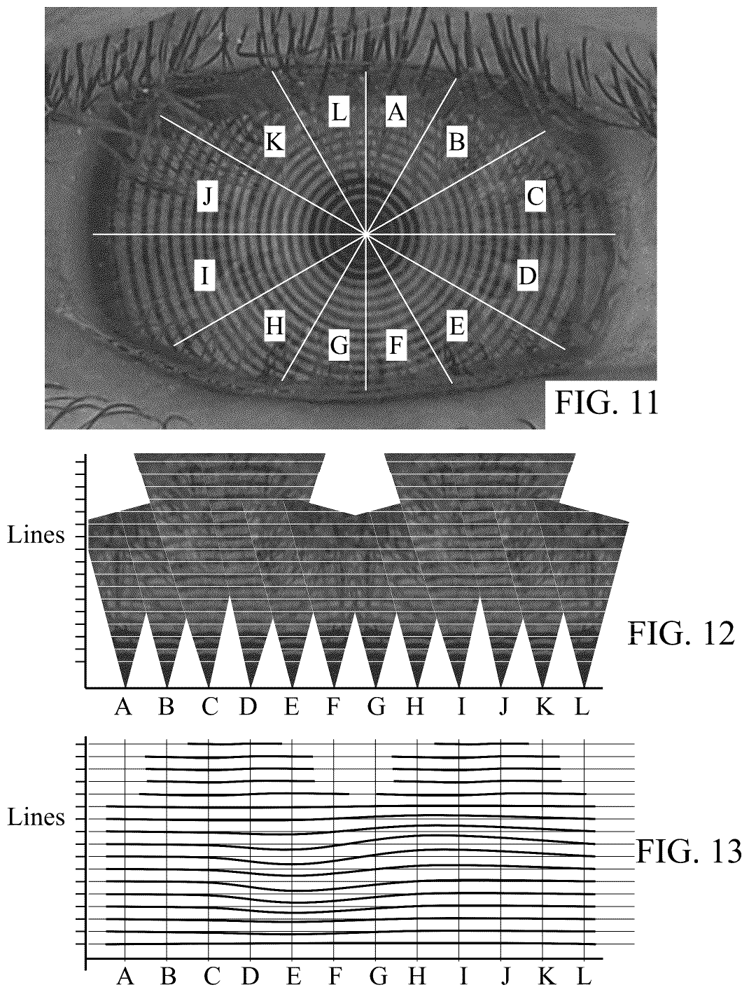

For simplicity of this disclosure the number of sectors in the image has been reduced to eight (8) angular sectors, shown as sectors A-L in this image. While the image is broken into 8 sectors, it could be broken into more than 8 sectors, depending upon the desired resolution. FIG. 11 shows the image from FIG. 9 with fewer angles of resolution with quadrant references. For processing the A-L quadrants are converted from cartesian to polar. FIG. 12 shows the image from FIG. 11 masked and converted from Cartesian to a polar graph. The centroid of each cornea sector is arranged at the zero location of the graph. This turns the circles into quasi-straight lines based upon the image. The converted polar image is divided into nonoverlapping blocks of equal size with the resulting graph in FIG. 13. FIG. 13 shows graph from FIG. 12 converted to lines for processing.

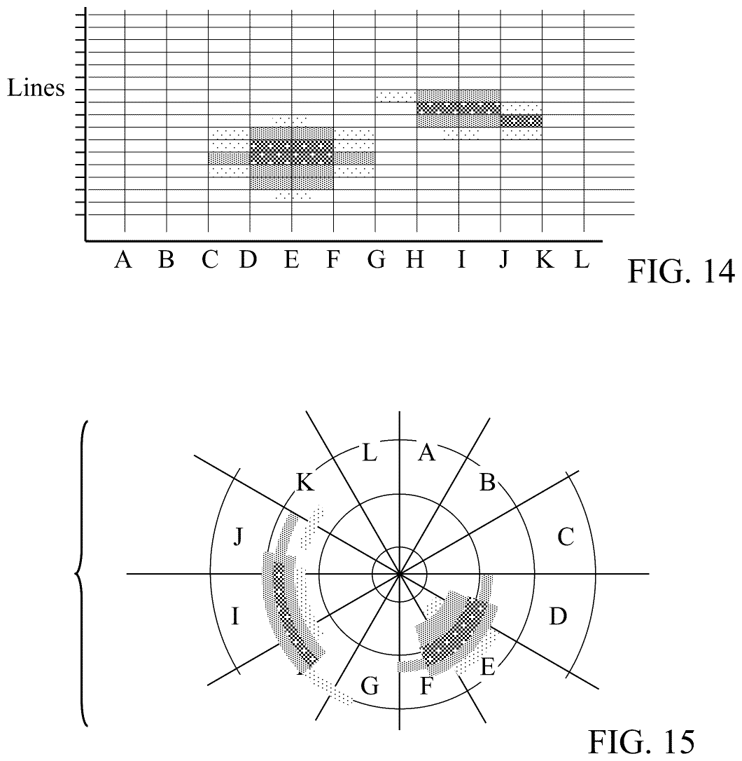

The deviation from straight lines is processed in blocks to generate the graph shown in FIG. 14. FIG. 14 shows the graph from FIG. 13 with block processing to approximate the deviation from normal using darker images or color to show the deviation and lighter, or a different color to show that the particular area is cornea is round. The polar graph can then the converted back to the cartesian appearance of the cornea.

FIG. 15 shows the block processing from FIG. 14 converted from Polar to Cartesian to show the imperfections in the cornea. Deviation from normal using darker images or color to show the deviation and lighter, or a different color to show that the particular area is cornea is round.

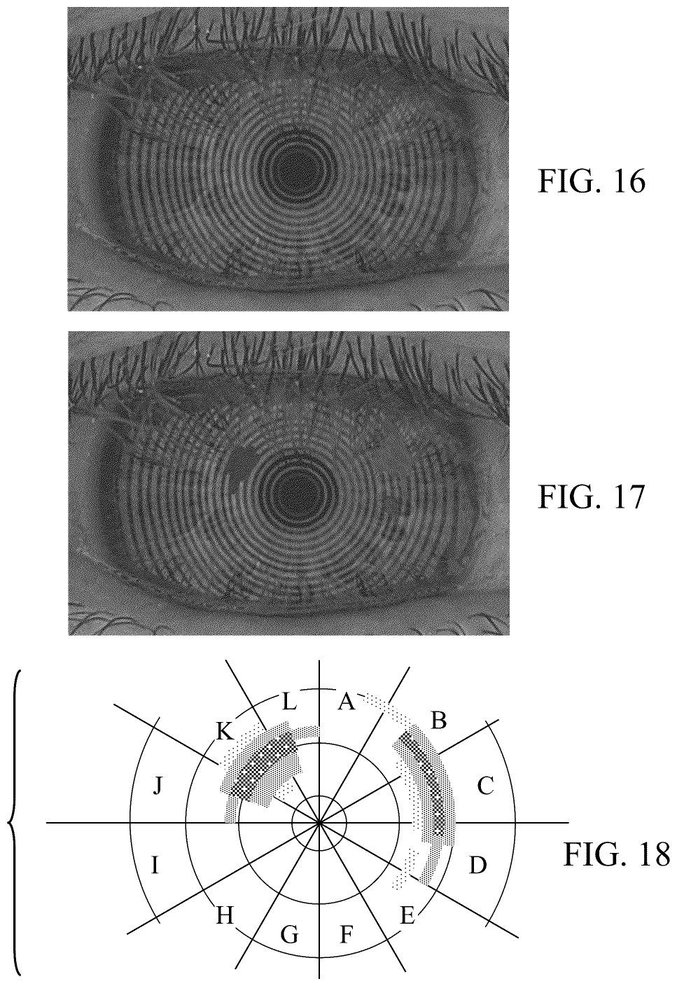

During the test the user is told to leave their eye open for 15 seconds without blinking. The smartphone can record a move or a series of images to determine if a blink has occurred and can reset the counter for the 15 second period. When a person has dry eyes, the dry area of the eye will stop reflecting a portion of the rings, or the rings will break-up over the 15 second time. FIG. 16 shows the initial cornea image and FIG. 17 shows the changed cornea image with tear film break-up. A comparison of the initial and the 15 second image can be compared, and the difference can be processed using a similar or the same masking, cartesian to polar, block processing and polar to cartesian conversion to result in the image shown in FIG. 18. FIG. 18 shows the tear film break-up after processing. This provides a record and storage of the tear film break-up.

Thus, specific embodiments of a smartphone Placido disc evaluation of tear film break-up device has been disclosed. It should be apparent, however, to those skilled in the art that many more modifications besides those described are possible without departing from the inventive concepts herein. The inventive subject matter, therefore, is not to be restricted except in the spirit of the appended claims.

1. A smartphone Placido disc evaluation of tear film break-up device comprising:
a clam shell clamping mechanism that is configured to clamp a mobile communication device with a camera within said clam shell clamping mechanism over said camera in said mobile communication device;
said clam shell clamping mechanism further includes an illuminating ring within a that surrounded by a reflective cone wherein light is directed away from said clam shell clamping mechanism and into said reflective cone;
said illuminating ring further includes a tube that extends from a first end of said illuminating ring within said reflective cone, and
said tube includes a plurality of light and dark rings whereby said light and dark rings project circular images in a curved surface placed at a second end of said tube.

2. The smartphone Placido disc evaluation of tear film break-up device according to claim 1, wherein said curved surface is an eye.

3. The smartphone Placido disc evaluation of tear film break-up device according to claim 1, wherein said second end of said tube has an eyepiece.

4. The smartphone Placido disc evaluation of tear film break-up device according to claim 1, wherein said first end of said tube has a centering orifice for centering said tube over said camera in said mobile communication device.

5. The smartphone Placido disc evaluation of tear film break-up device according to claim 1, wherein said light and dark rings are sized and spaced based upon the radius of curvature of said curved surface.

6: The cell phone cornea Placido imaging according to claim 1, wherein said illuminating ring has more than one level of brightness.

7. The smartphone Placido disc evaluation of tear film break-up device according to claim 1 further includes determining an image size by counting the number of pixels across said second end of said tube.

8. The smartphone Placido disc evaluation of tear film break-up device according to claim 7 further includes scaling said image size with a diameter of said second end of said tube to determine an image resolution.

9. The smartphone Placido disc evaluation of tear film break-up device according to claim 8 further includes using said resolution to determine a tear meniscus.

10. The smartphone Placido disc evaluation of tear film break-up device according to claim 8 further includes using said image to determine a roundness of said curved surface.

11. The smartphone Placido disc evaluation of tear film break-up device according to claim 10 wherein said roundness is determined by determining a center of said curved surface.

12. The smartphone Placido disc evaluation of tear film break-up device according to claim 11 further includes centering a grid on said center of said image.

13. The smartphone Placido disc evaluation of tear film break-up device according to claim 12 further includes defining edges of said plurality of light and dark ring.

14. The smartphone Placido disc evaluation of tear film break-up device according to claim 13 further includes using said edges to determine said roundness and a concentricity of said curved surface.

15. The smartphone Placido disc evaluation of tear film break-up device according to claim 14 wherein said roundness and said concentricity are determined by separating said image into at least 8 angular sectors.

16. The smartphone Placido disc evaluation of tear film break-up device according to claim 15 further includes converting said at least 8 angular sectors from cartesian to a polar image.

17. The smartphone Placido disc evaluation of tear film break-up device according to claim 16 further includes converting said polar image into nonoverlapping blocks of equal size.

18. The smartphone Placido disc evaluation of tear film break-up device according to claim 17, further includes processing said nonoverlapping blocks of equal size and determining deviation.

19. The smartphone Placido disc evaluation of tear film break-up device according to claim 18, further includes converting said deviation in said polar image into a processed cartesian image that shows imperfections of said roundness of said curved surface.

20. The smartphone Placido disc evaluation of tear film break-up device according to claim 19, further includes using said processed cartesian image to determine a tear film break-up.
